(19) United States
(12) Patent Application Publication
(10) Pub. No.: US 2021/0077023 A1
(43) Pub. Date: Mar. 18, 2021

(54) MICROCIRCULATION ASSESSMENT DEVICE

(71) Applicants: JAMES CONNORS, Broadview Heights, OH (US); CRAIG VERDIN, North Royalton, OH (US); NILIN RAO, Parma Heights, OH (US)

(72) Inventors: JAMES CONNORS, Broadview Heights, OH (US); CRAIG VERDIN, North Royalton, OH (US); NILIN RAO, Parma Heights, OH (US)

(73) Assignee: KENT STATE UNIVERSITY, Kent, OH (US)

(21) Appl. No.: 16/611,792

(22) PCT Filed: May 10, 2018
(86) PCT No.: PCT/US2018/032072

§ 371 Date: Nov. 7, 2019

Related U.S. Application Data
(60) Provisional application No. 62/504,747, filed on May 11, 2017.

(51) Int. Cl. A61B 5/00; A61B 5/026; A61B 5/1455; A61B 5/0205; G16H 40/67; G16H 50/30; G16H 30/40; G16H 50/50

(52) U.S. Cl. CPC A61B 5/6807; A61B 5/0261; A61B 5/0002; A61B 5/742; A61B 5/14552; G16H 20/40; G16H 40/67; G16H 50/30; G16H 30/40; G16H 50/50; A61B 2562/046; A61B 5/0205

(57) ABSTRACT

A microcirculation assessment device includes a wearable garment that includes one or more hyperspectral sensors. The hyperspectral sensors are positioned adjacent to the skin of a wearer of the garment. Such sensors enable an assessment of the macro and micro vasculature of a patient to be obtained, so as to facilitate medical diagnosis.

### CROSS-REFERENCE TO RELATED APPLICATION

This application claims the benefit of U.S. Provisional Application No. 62/504,747 filed May 11, 2017, the contents of which are incorporated herein by reference.

### TECHNICAL FIELD

Generally, the embodiments disclosed herein relate to medical assessment devices. In particular, the embodiments disclosed herein relate to devices that assess the macrovasculature and the microvasculature status of various portions of a patient's body. More particularly, the embodiments disclosed herein relate to microcirculation assessment devices that utilize sensors that are provided on a patient wearable garment.

### BACKGROUND

Several comorbidities can negatively impact the care of patients that have foot and ankle aliments. For example, in the case of peripheral arterial disease (PAD), the lack of quality blood flow to the lower extremities of a patient's body increases the complication rate of this disease substantially. In addition, failing to assess blood circulation at the site of an incision, in the case of simple in-office procedures or complex surgeries, increases the potential for wound healing complications and post procedural infections to occur. As an understanding of the arterial distribution of the lower extremities has been acquired, insight into the vascular anatomy of the foot and ankle region has been elucidated. As a result of this increased understanding, the need for considering a patient's macrovasculature is now clearly evident when planning medical procedures and surgical incisions.

Furthermore, the ability to optimize outcomes in limb salvage, surgical procedures, and wound care, decreases potential complications in a multifactorial diseased patient population. For example, given that many patients within the podiatric community present with significant comorbidities, such as diabetes, peripheral neuropathy, cardiovascular disease, critical limb ischemia (CLI) and peripheral vascular disease (PVD), it is imperative that physicians are able to intervene in a timely manner to take preventative action to reduce the risk for adverse outcomes, such as infection, which may require hospitalization, amputation, and even death. The contribution of peripheral arterial disease (PAD) alone to adverse patient outcomes has been evaluated, and was found to significantly increase the risk of amputation, while a concurrent infection in such an area with impaired arterial blood flow quadrupled such risk. This result is attributed largely to the inability of oral, or even parenteral, antibiotics to reach the distal extremity site of infection due to inadequate arterial flow to the infected area.

One technique used to obtain a real-time assessment of a patient's microvasculature and associated collateral circulation during a medical or surgical procedure is angiography. Angiography, however, requires the use of a radiopaque contrast medium or agent that is invasively injected into the arterial tree of a patient, whereupon the arterial tree is imaged using radiography techniques. Furthermore, the use of angiography requires expensive hospital-based equipment, certified specialists, and is generally, due to its nature, inconvenient to the patient. Moreover, angiography may be unnecessarily performed due to false positives that arise with the use of current vascular assessment techniques.

In addition to angiography, non-invasive vascular studies have been utilized as a tool to initially assess arterial blood supply in the lower extremities of patients. For example, an ankle-brachial pressure index (ABI) has been used extensively to identify the severity of peripheral arterial disease (PAD), with an ABI value of less than 0.9 indicating the presence of disease. In critical limb ischemia (CLI), ABI values of less than 0.5 correspond with an increasingly poor prognosis. While advantageous to assess arterial blood flow to the ankle, ABI lacks the ability to evaluate microvascular circulation. Furthermore, ABI can be artificially inflated in the case of calcified arteries, thereby leading to erroneous results. To better assess vascular supply to the entire foot, toe-brachial pressure index (TBI) and transcutaneous partial pressure of oxygen (TcPO2) have been employed as part of further non-invasive vascular testing techniques. While TBI may be rendered obsolete or invalid if an ulcer is located on a lesser digit or toes of a patient, or in a non-corresponding anatomic location of a patient, TcPO2 has proven to be an adequate measure of ulcer healing potential. It has also been found that patients with diabetic foot ulcers had a poor healing rate with a TcPO2 of less than 30 mmHg, and that revascularization of patients with a TcPO2 of less than 30 mmHg demonstrated that following physician intervention, their healing rates were comparable to patients with a TcPO2 that is greater than 30 mmHg. Unfortunately, in the case of assessing the plantar surface of a patient's foot, TcPO2 measurements are generally inaccurate due to increased skin thickness, and due to the characteristics of the foot in obese patients. In addition, both TBI and TcPO2 require an off-site location that has skilled technicians that are capable of performing the specialized diagnostic procedure on a patient. Thus, it would be desirable to have an easy to use device that is capable of being used in a doctor's office to accurately measure microvascular arterial supply of a patient, including all aspects of the foot and ankle, including the digits, for example.

Tissue oxygen saturation (stO2) monitoring provides a potential diagnostic tool that can be easily used in a physician's office for assessing microvascular circulation in the extremities of a patient. Also known as a tissue spectrometer, stO2 utilizes near-infrared spectroscopy (NIRS) to measure chromophore compounds, such as total hemoglobin, oxyhemoglobin and deoxyhemoglobin concentrations. Specifically, during an stO2 analysis, light is absorbed, reflected or scattered during the process of illuminating the chemical components of the blood, whereupon the different wavelengths of the reflected or scattered light are analyzed. This technology uses a laser diode to protect a near-infrared light beam (700 nm-1000 nm) that penetrates into a surface of a patient's skin at a depth of about 8 centimeters (cm), as compared to visible light that is limited to a penetration depth of about 1 cm. NIRS that is utilized by stO2 has traditionally been used in hospital settings to evaluate overall tissue oxygenation by placing the probe over the thenar eminence of a patient's hand. However, due to the nature of the foot, such techniques have not been able to be applied successfully thereto.

In addition, while there are non-invasive devices that aim to assess peripheral macrovascular status through various means, such devices fail to effectively assess microvascular status. Given that the vast majority of patients in the podiatric community suffer from diabetes and other comorbidities, the need for true microvascular assessment is crucial for quality patient care. Currently, podiatrists heavily utilize the ankle-brachial index/toe brachial index, and various visual cues to assess a patient's vascular status. However, these techniques do not provide sufficient measurement data of the microvasculature and tissue oxygenation of the entire foot of the patient. Oxygen plays an important role in wound healing, as it is essential to biological functions, such as cell proliferation, immune responses and collagen synthesis. Poor oxygenation is directly associated with the development of non-healing ischemic wounds, which affect more than 6 million people each year in the United States alone at an estimated cost of about $25 billion dollars. Thus, it would be desirable to have an easy to use device for use in the physician's office that is capable of measuring tissue oxygenation of a patient, including in the region of the foot and ankle.

Therefore, there is a need for a medical device that provides a clinician with information about a patient's metabolic activities, oxygen distribution, and perfusion at the macroscopic and microscopic level in an effort to provide information that can be used to optimize outcomes in patient interventions. Knowledge and visualization of a patient's macrovasculature and microvasculature that are enabled by such a device can guide the clinician during intervention efforts, which ultimately reduces healing, time, avoids complications and encourages positive outcomes in patient care. There is also a need for a microcirculation assessment device that provides a medical professional, such as a foot and ankle clinician, with an accurate real-time vascular assessment tool, which may be utilized in the physician's office, as opposed to a hospital. This is desirable, as existing in-office physician practices are currently limited to performing macrovascular assessments, which lack reliability due to several factors, such as patient obesity and skin thickness in the foot region. Furthermore, in order to carry out in-office vascular testing with increased reliability requires additional patient appointments, travel and added costs, which are unwanted.

### SUMMARY

It is one aspect of the various embodiments disclosed herein to provide a microcirculation assessment device that is a portable device containing strategically placed near-infrared (NIR) hyperspectral probes or sensors, and oxymetry probes or sensors as part of a garment that can be worn on a patient's foot or other portion of their body. These probes or sensors are placed in areas that correspond with areas of macrovasculature and collateral blood circulation, including areas on the human foot. By providing sufficient information on both macrovascular and microvascular status, a physician can intervene in a patient's care using the acquired knowledge, so as to facilitate healing and avoid possible complications in the targeted treatment area. The corresponding vascular assessment may be transmitted to a display to provide a computer generated (virtual) visual model, of the macrovasculature and/or microvasculature of the patient, such as in 2 or 3 dimensions, for physician visualization, as well as patient education. The visual model can be used as a tool to track changes that may be occurring in the patient's body, such as their foot, over the course of time. This provides surgeons and physicians with vital data to facilitate medical procedure planning, revascularization assessment, as well as ulcer and wound healing potential and progression.

Another aspect of the various embodiments disclosed herein is to provide a microcirculation assessment device comprising a wearable garment; and one or more near-infrared hyperspectral sensors carried by the garment.

It is yet another aspect of the various embodiments disclosed herein to provide a method of analyzing microcirculation of a person, the method comprising providing a wearable garment carrying one or more near-infrared hyperspectral sensors; wearing the wearable garment so that the one or more sensors are adjacent to skin of the person; emitting, from the one or more sensors, near infrared light into the skin; detecting, at the one or more sensors, the infrared light that has been reflected back from the skin, as detected infrared light; and computing one or more of oxygen saturation, oxyhemoglobin, and deoxyhemoglobin based on the detected infrared light.

### BRIEF DESCRIPTION OF THE DRAWINGS

These and other features and advantages of the various embodiments disclosed herein will become better understood with regard to the following description, appended claims, and accompanying drawings wherein:

FIG. 1 is a perspective view of a microcirculation assessment device in accordance with one or more embodiments disclosed herein;

FIG. 2 is another perspective view of the microcirculation assessment device in accordance with one or more embodiments disclosed herein;

FIG. 3 is another perspective view of the microcirculation assessment device in accordance with one or more embodiments disclosed herein;

FIG. 4 is another perspective view of the microcirculation assessment device in accordance with one or more embodiments disclosed herein; and

FIG. 5 is a perspective view of a display utilized by the microcirculation assessment device in accordance with one or more embodiments disclosed herein.

### DETAILED DESCRIPTION

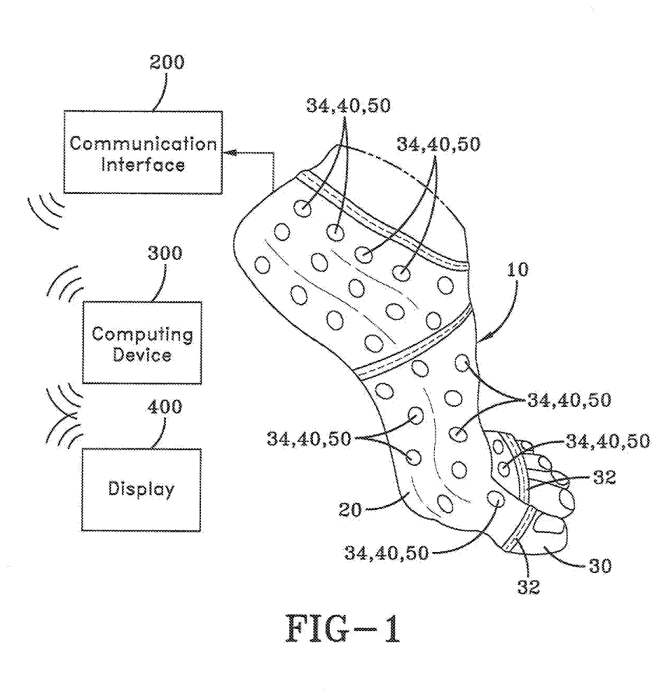

A microcirculation assessment device 10 is shown in FIG. 1 of the drawings. The device 10 includes appropriate sensing, communication and display capabilities to carry out the microcirculation analysis functions to be discussed.

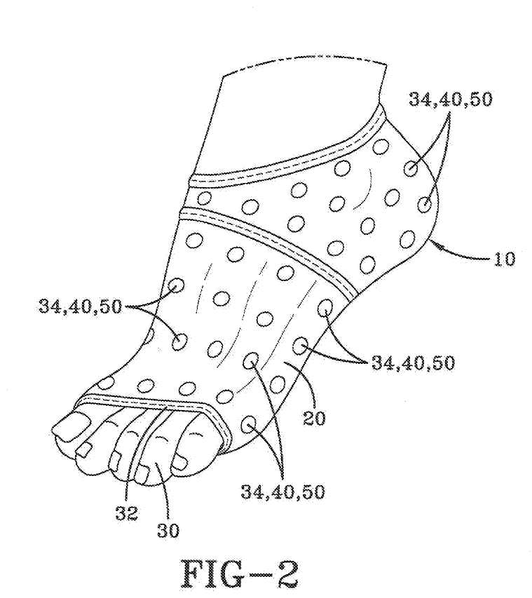

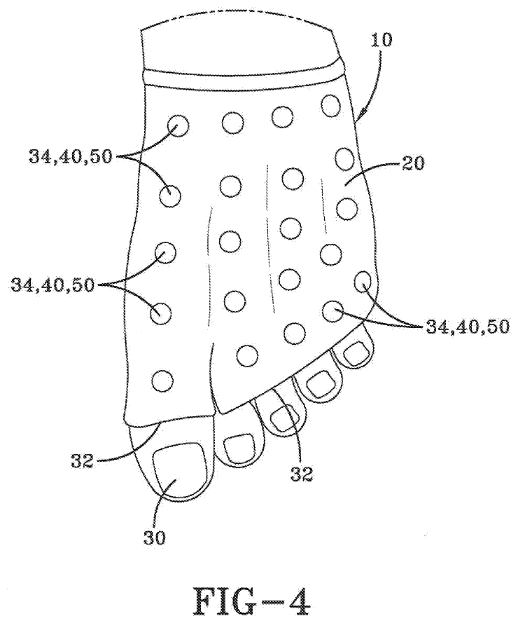

In particular, the device 10 includes a garment 20 that is suitable to be worn by any portion of a patient's body, or extremity thereof, such as their leg, foot, arm, or hand for example. The garment may comprise a sleeve, shirt, pants or the like. However, for the purposes of the discussion herein, the garment 20 is configured as a sock. In the case of the sock, it can be worn by a patient's foot 30, as shown in FIGS. 1-4. It should be appreciated that the sock may include one or more openings 32 proximate to a patient's toes, allowing their toes to extend there through, or the sock may be entirely closed preventing the toes from extending through the end of the sock. In other embodiments, the sock may include one or more toe sleeves that fully or partially cover one or more toes, or portion thereof, of the patient. In some embodiments, the sock may essentially be a sleeve that is able to be carried upon some portion of the foot 30. It should also be appreciated that the garment 20 may be formed of any suitable material, including material that is resilient, stretchable, or conformable, as well as any combination thereof, so as to be suitably retained to the portion of the patient's body to which it is attached. The garment 20 may also include adjustment or closure devices, such as straps or hook and loop fasteners for example to further secure the garment 20 to the patient.

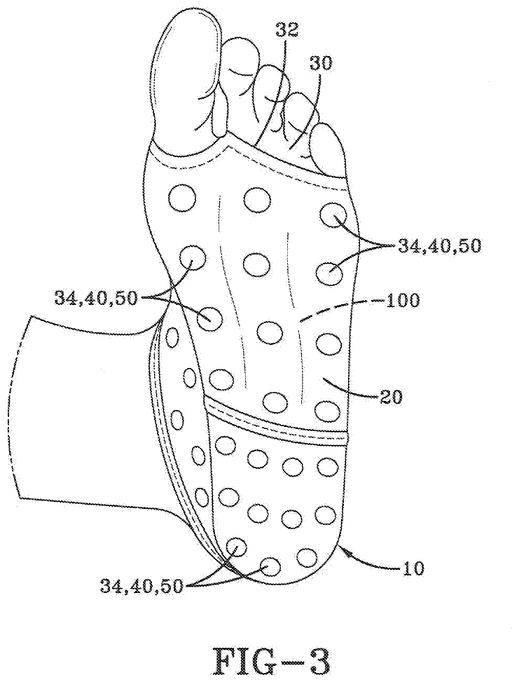

The device 10 also includes one or more near-infrared (NIR) hyperspectral sensors 34 that are carried by the garment 20. Each of the hyperspectral sensors 34 includes one or more hyperspectral emitters 40, and one or more hyperspectral optical detectors 50. As such, when the garment 20 is worn by a patient, the emitter 40 and the detector 50 of a given sensor 34 are positioned so that they are adjacent to, at least proximate or near to, or in physical contact with, a surface of a patient's skin in the region of their body that is under assessment. In one embodiment, the hyperspectral sensors 34 may be positioned on a surface of the sock forming the garment 20, so that when the sock is worn by a patient, the emitter 40 and detector 50 are positioned adjacent or proximate to, and in some cases in contact with, a plantar aspect or surface 100 of their foot 30 (i.e. sole of their foot), as shown in FIG. 3. Thus, when the garment 20 is worn by the patient, the one or more near-infrared (NIR) hyperspectral sensors 34 operate to measure various biological parameters of a target tissue, such as soft tissue, such as the tissues of a human foot. Such biological parameters may include, but are not limited to: oxygen saturation, oxyhemoglobin, and deoxyhemoglobin.

For example, in some embodiments, one or more hyperspectral emitters 40 are configured to emit a near infrared (IR) light beam having a wavelength of about 700 nm to 1000 nm, which penetrates the patient's soft target tissue to a suitable depth. It should be appreciated that in some embodiments of the device 10, a plurality of light beams may be emitted by one or more emitters 40 each having a wavelength within one or more sub-bands of the 700 nm to 1000 nm band and/or within one or more sub-bands of the 2500 nm to 15000 nm band, and/or one or more sub-bands of another wavelength band. Specifically, the emitted light interacts with the soft target tissue, whereupon the emitted light is transmitted through or into the tissue. The light received in the tissue is then reflected by the tissue or absorbed by the tissue, as well as combinations thereof, at various amounts depending on the particular biological characteristics existing in the tissue under investigation. One or more hyperspectral optical detectors 50, which are sensitive to the near infrared (IR) wavelength of about 700 nm to 1000 nm, are then utilized to detect the portion of the emitted light that is reflected back from the target tissue. This reflected light is then analyzed to measure the percent of chromophore absorption that was caused by the target tissue using known techniques. In addition, the light detected by the hyperspectral optical detector 50 may be further analyzed to obtain various biological characteristics associated with the microvasculature and microvasculature of the patient.

In some embodiments, the reflected light from the target tissue of a patient as captured by the detector 50 may be used to calculate a ratio of oxygenated and deoxygenated hemoglobin of a patient using the Beer-Lambert Law. Such ratio calculation is enabled by the fact that light transmittance out of the target tissue of the patient is related to the optical depth and light absorbance of the target tissue under investigation. For example, light transmittance associated with a material sample equals the radiant flux transmitted divided by the radiant flux received. If “lo” is the intensity of light entering a solution and “lt” is the intensity of light exiting the solution/material, such as a patient's soft tissue, then the light transmittance, T, of the solution/material is given as lt/lo. Light transmittance is also expressed as a percentage by the following equation: (lt/lo)*(100). In some cases, the light absorbance, A, rather than light transmittance, may be used for the amount of light a solution/material absorbs. Light absorbance is defined by the equation A=−log(T) or A=log(lo/lt). The light absorbance of a solution/material depends on the quantity of light that is absorbed by the species in the solution/material, the wavelength of the light entering the solution/material, the length of the solution/material the light has to pass through, and the concentration of the solution/material.

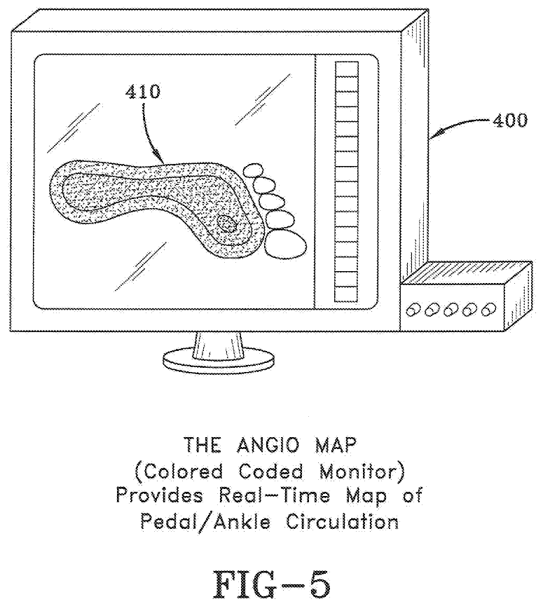

Alternatively, this ratio of oxygenated and deoxygenated hemoglobin may also be converted to a visual image based on the locations of the sensor 34 on the foot or ankle of the patient, such as in the case of the virtual or graphical model 410, shown in FIG. 5, which is presented on a viewable display 400. Furthermore, as blood flow in the tissue under investigation becomes progressively impaired, the light absorbance measurement curve will decrease. This indicates that more or less light will be absorbed, and may be displayed as a color change (e.g. gradient) on a corresponding anatomic location of a model of a patient's body, such as the model of their foot 410 for example. It should be appreciated that in some embodiments, the sensors 34 may utilize stO2 monitoring through NIRS to measure blood flow in a portion of a patient's body, such as their foot and ankle region.

The device 10 may also include a communication interface 200, as shown in FIG. 1, which is configured to communicate data acquired by the sensors 34, using a wired or wireless communication protocol, to any suitable computing device or data storage device 300 or combination thereof. For example, the computing/storage device 300 may communicate with the interface 200 using cloud computing technology, as well as BLUETOOTH or WIFI, or any other suitable communication protocol. In some embodiments, the computing device or data storage device 300 may include a cloud-based computer or other cloud-enabled data storage device. In some embodiments, the data acquired by the computing or data storage device 300 may be utilized, and in some cases may be further processed, to generate images for presentation on the display 400 that are based on the data acquired by one or more of the sensors 34. For example, the computing device 300 may process the data acquired from one or more sensors 34 to generate a virtual or graphical computer model, rendering, or image in 2 or 3 dimensions that is representative of the macrovasculature and/or microvasculature of the patient for presentation on the display 400. Furthermore, such sensor data may be further combined with other data to generate additional images, models, graphs, overlays and charts for presentation on the display 400. It should be appreciated that the display 400 may comprise any suitable display, such as on LCD (liquid crystal display) for example. Additionally, in other embodiments, the communication interface 200 may be configured to communicate through any suitable wired or wireless protocol directly with the display 400, so as to present images associated with, or based on, the data acquired by the sensors 34 without processing or storage by the computing/storage unit 300. It should be appreciated that the acquisition of data from the sensors 34 and/or the display of images based on such data, may be performed in real-time or near real-time, and that the displayed images may be presented as static or still images, or may be presented as dynamic or moving images, as well as combinations thereof. It should be appreciated that communication between the communication interface 200, the computing/storage unit 300, and the display 400 may be carried out using any desired communication path and any combination of wireless and wired communication protocols. In other embodiments, the communication interface 200 may include a data storage device, such as a fixed or portable/removable data storage device (e.g. flash memory card) that records the data acquired from the sensors 34 for subsequent transfer. Such data may then be later transferred therefrom by the communication interface 200, or alternatively, in the case of the portable data storage device, removed and placed in communication with a computing unit for subsequent data transfer and data analysis and imaging.

The display 400, shown in FIGS. 1 and 5, may comprise any suitable display capable of displaying images in 2 or 3 dimensions, such as a liquid crystal display (LCD). For example, with regard to 3-dimensional images, the display 400 may be configured to render images in a 2 dimensional format, with such images being formatted to give the appearance that they are in 3-dimensions. In addition, the display 400 may be configured to render images so that they are presented in a 3-dimensional format when viewed with or without specialized 3D-glasses. In some embodiments, the display 400 may comprise a head-mounted virtual-reality or augmented reality display unit. It is also contemplated that the display 400 is capable of displaying images in real-time, or in near real-time, as the data associated with the displayed image is collected by the sensors 34. In still further embodiments, the generated sensor data, or images derived therefrom, may be further modified, overlaid or superimposed with other data, images or visual models (2D or 3D), such as other biological structures for example, to facilitate and enhance the assessment capabilities of a diagnostic professional, such as in the case of augmented reality for example.

Thus, a diagnostic professional utilizing the device 10 is provided, in some embodiments, with images of a patient's vasculature that are acquired through the use of near-infrared spectroscopy and hyperspectral imaging provided the by the emitter 40 and the detector 50 of the sensor 34. As previously discussed, such imaging can be applied to the lower extremities of a patient, such as their leg or foot, to analyze their vasculature non-invasively by acquiring information about the metabolic state of targeted tissue being analyzed by measuring light absorption of blood-borne molecules therein, such as oxyhemoglobin and deoxyhemoglobin. In some embodiments, the near-infrared hyperspectral imaging that is performed by the sensor 34 is characterized by relatively short wavelengths of about 2500 nm to 15000 nm, however in other embodiments, other wavelengths may be used, such as about 700 nm to 1000 nm previously discussed. However, it should be appreciated that other suitable wavelengths may be used. In addition, in some embodiments, the display 400 may produce color-coded images associated with the computer generated model 410 of the patient's body, such as their foot, as shown in FIG. 5, that reflect the level of tissue oxygenation within the targeted tissue region imaged by the sensors 34. This provides a physician with the ability to visually quantify the arterial supply of both large and small vessels, as well as collateral circulation in the targeted portion of the body.

It should be appreciated that the light emitter 40 and the light detector 50 may be separated by various distances, such as between 2.5 to 3.0 cm for example. In some embodiments, the light emitter 40 and the light detector 50 may each individually operate to enable dual operation of emitting light and detecting light. In other words, the emitter 40 may be configured to both emit light to the tissue and detect light received back from the tissue. Similarly, the detector 50 may be configured so that it is capable of emitting light to the tissue and detecting light received back from the tissue. It should be appreciated that in some embodiments, the light emitter 40 generates a near infrared light that has a wavelength of about 700 nm to 1000 nm that penetrates the tissue in a shallow arc.

Accordingly, one advantage of the various embodiments disclosed herein is that a microcirculation assessment device is able to assess the vasculature of a patient in a cost-effective manner. Another advantage of the various embodiments disclosed herein is that the microcirculation assessment device is able to quickly assess the microvascular blood flow, such as in an entire foot and ankle regardless of the thickness of the plantar skin or due to patient obesity. Still another advantage of one or more embodiments disclosed herein is that a microcirculation assessment clinician is able to reduce or avoid the occurrence of potential complications, delays and patient non-compliance, which can have a significant negative impact on expenditures and resources of a patient. Another advantage of one or more embodiments disclosed herein is that the microcirculation assessment device provides a physician with a user-friendly assessment tool that is capable of providing robust assessment features that can be performed on an expedited basis, independently of the particular facility, location or resources that are provided thereby. Yet another advantage of various embodiments disclosed herein is that the microcirculation assessment device provides visual information to the physician or other diagnostic professional that is able to impact patient intervention in an effort to optimize outcomes in the case of wound healing, limb salvage, and surgical intervention.

Therefore, it can be seen that the objects of the various embodiments disclosed herein have been satisfied by the structure and its method for use presented above. While in accordance with the Patent Statutes, only the best mode and preferred embodiments have been presented and described in detail, with it being understood that the embodiments disclosed herein are not limited thereto or thereby. Accordingly, for an appreciation of the true scope and breadth of the embodiments, reference should be made to the following claims.

1. A microcirculation assessment device comprising:
a wearable garment; and
one or more near-infrared hyperspectral sensors carried by said garment, wherein each one of said sensors includes a hyperspectral light emitter and a hyperspectral light detector that are arranged laterally adjacent to each other.

2. The device of claim 1, further comprising:
a communication interface coupled to said one or more near-infrared hyperspectral sensors;
wherein said communication interface is configured to communicate data therefrom that is acquired by said near-infrared hyperspectral sensors.

3. The device of claim 2, further comprising:
a display device configured to receive said data from said communication interface and to generate one or more images based on said data.

4. The device of claim 3, wherein said one or more images are associated with a level of tissue perfusion and oxygenation.

5. The device of claim 3, wherein said one or more images include a large arterial vessel image, a small arterial vessel image, or a collateral circulation image, and combinations thereof.

6. The device of claim 3, wherein said one or more images are in three-dimensions.

7. The device of claim 1, wherein said garment is a sock.

8. The device of claim 7, wherein said one or more near-infrared hyperspectral sensors are positioned relative to said sock so as to be capable of being positioned adjacent to a portion of skin of a person.

9. The device of claim 8, wherein said portion of the skin is a plantar surface of a foot.

10. (canceled)

11. The device of claim 1, wherein said light emitter emits light having a wavelength from about 700 nm to 1000 nm.

12. The device of claim 1, wherein said light emitter emits light having a wavelength from about 2500 nm to 15000 nm.

13. A method of analyzing microcirculation of a person, the method comprising:
providing a wearable garment carrying one or more near-infrared hyperspectral sensors, wherein each one of said sensors includes a hyperspectral light emitter and a hyperspectral light detector that are arranged laterally adjacent to each other;
wearing said wearable garment so that said one or more sensors are adjacent to skin of the person;
emitting, from said one or more sensors, near infrared light into the skin;
detecting, at said one or more sensors, said infrared light that has been reflected back from the skin, as detected infrared light; and
computing one or more of oxygen saturation, oxyhemoglobin, and deoxyhemoglobin based on said detected infrared light.

14. The method of claim 13, wherein said garment is a sock.

15. The method of claim 13, further comprising:
displaying an image based on one or more of said oxygen saturation, oxyhemoglobin, and deoxyhemoglobin.

16. The method of claim 15, wherein said image is three-dimensional (3D).

17. The method of claim 13, wherein said one or more sensors are positioned adjacent to the skin on a plantar surface of a foot.

18. (canceled)

19. The method of claim 13, wherein said infrared light emitted by said light emitter at said emitting step has a wavelength from about 700 nm to 1000 nm.

20. The method of claim 13, wherein said infrared light emitted by said light emitter at said emitting step has a wavelength from about 2500 nm to 15000 nm.
